(19) United States
(12) Patent Application Publication
(10) Pub. No.: US 2024/0120545 A1
(43) Pub. Date: Apr. 11, 2024

(54) IONIC LIQUID BASED ON BIS(FLUOROSULFONYL)IMIDE

(71) Applicant: ARKEMA FRANCE, COLOMBES (FR)

(72) Inventor: Gregory SCHMIDT, Pierre-Benite Cedex (FR)

(73) Assignee: ARKEMA FRANCE, COLOMBES (FR)

(21) Appl. No.: 18/270,325

(22) PCT Filed: Dec. 15, 2021
(86) PCT No.: PCT/FR2021/052343

§ 371 Date: Jun. 29, 2023

(30) Foreign Application Priority Data
Jan. 4, 2021 (FR) FR2100021

(51) Int. Cl. H01M 10/0568; H01M 10/0525

(52) U.S. Cl. CPC H01M 10/0568; H01M 10/0525

(57) ABSTRACT

The invention relates to an ionic liquid comprising an anion of formula (I) and at least one onium cation, said ionic liquid having a colour of less than 115 Hazen units on the APHA scale. The invention also relates to a method for purifying an ionic liquid comprising an anion of formula (I) and at least one onium cation.

### FIELD OF THE INVENTION

The present invention relates to ionic liquids comprising the bis(fluorosulfonyl)imide (FSI) anion which are suitable for use as the electrolyte in a battery, and to processes for purifying ionic liquids.

### TECHNICAL BACKGROUND

Lithium (Li) batteries, such as lithium-ion batteries, are commonly used in electric vehicles and in mobile and portable devices.

A lithium-ion battery or a lithium-sulfur battery comprises at least a negative electrode (anode), a positive electrode (cathode), an electrolyte and preferably a separator. The electrolyte generally consists of a lithium salt dissolved in a solvent which is generally a mixture of organic solvents, in order to have a good tradeoff between the viscosity and the dielectric constant of the electrolyte.

Additives can be added to improve the stability of the electrolyte salts or passivation layers. Indeed, the passivation layers formed during the first charge/discharge cycles of a battery are vital to the longevity of the battery. Passivation layers that may be mentioned include in particular the passivation of aluminum, which is generally the current collector used at the cathode, and the solid-electrolyte interface (SEI), which is the inorganic and polymeric layer that is formed at the anode/electrolyte and cathode/electrolyte interfaces. The stability of these interfaces is a substantial challenge for improving battery lifetime.

Another major challenge is improving overall battery safety, especially for electric vehicle applications. Indeed, the flammability of the solvents used in the electrolytes is a problem. Various solutions exist for avoiding the flammability of the electrolyte, such as the use of fluorinated solvents or of ionic liquids.

The use of fluorinated solvents has the drawback of reducing the ionic conductivity of the electrolyte. Ionic liquids do not have this drawback; however, substantial amounts of ionic liquid must be used to make the electrolyte nonflammable. In these conditions, it is particularly vital to use ionic liquids that exhibit good electrochemical stability, to obtain batteries of sufficient longevity.

Ionic liquids can also be used in other applications besides lithium batteries.

Document WO 2016/049391 describes ionic liquids, especially for the treatment and cleaning of surfaces.

Document WO 99/40025 concerns low-melting-point ionic compounds in which the cation is an onium cation and the anion comprises an imide ion.

There is therefore a genuine need to provide ionic liquids of improved electrochemical stability, so making it possible to obtain batteries having an enhanced lifetime.

### SUMMARY OF THE INVENTION

The invention relates first to an ionic liquid comprising an anion of formula (I):

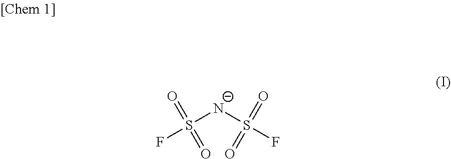

and at least one onium cation,
said ionic liquid having a color of less than 115 Hazen units on the APHA scale.

In some embodiments, the onium cation is a quaternary ammonium ion, a pyridinium ion, an imidazolium ion, an oxazolidinium ion, a piperidinium ion, and/or a phosphonium ion.

In some embodiments, the ionic liquid has a color of less than or equal to 100 Hazen units, preferably less than or equal to 75 Hazen units, more preferably less than or equal to 50 Hazen units, still more preferably less than or equal to 25 Hazen units and even more preferably less than or equal to 20 Hazen units, on the APHA scale.

In some embodiments, the ionic liquid consists essentially of the anion of formula (I) and the onium cation.

In some embodiments, the ionic liquid further comprises from 0 to 20 ppm of F⁻ ions, from 0 to 20 ppm of Cl⁻ ions, from 0 to 50 ppm of SO₄²⁻ ions, from 0 to 20 ppm of Na⁺ ions and from 0 to 20 ppm of K⁺ ions.

The invention also relates to a process for purifying an ionic liquid, comprising the following steps:
supplying an initial ionic liquid comprising an anion of formula (I):

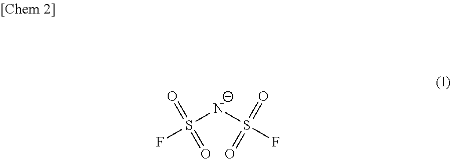

and an onium cation;
contacting said initial ionic liquid with activated carbon, to collect a decolorized ionic liquid;
at least once aqueously washing the decolorized ionic liquid;
collecting a purified ionic liquid.

In some embodiments, the initial ionic liquid has a color of greater than or equal to 115 Hazen units on the APHA scale.

In some embodiments, the purified ionic liquid has a color of less than 115 Hazen units on the APHA scale.

In some embodiments, the initial ionic liquid is in solution in a polar organic solvent, preferably selected from the group consisting of esters, ethers, nitriles, carbonates and mixtures thereof.

In some embodiments, the activated carbon has a specific surface area of greater than or equal to 300 m²/g.

In some embodiments, the mass ratio of the activated carbon relative to the initial ionic liquid is from 0.05 to 0.5.

In some embodiments, the aqueous washing comprises contacting the decolorized ionic liquid, dissolved in a water-insoluble polar organic solvent, with a mass of demineralized water.

The invention also relates to an ionic liquid obtainable by the process as described above.

The invention also relates to an electrochemical cell comprising a negative electrode, a positive electrode and an electrolyte, wherein the electrolyte comprises an ionic liquid as described above.

The invention also relates to a battery comprising at least one electrochemical cell as described above.

The present invention makes it possible to meet the need expressed above. It provides more particularly an ionic liquid exhibiting improved electrochemical stability, which can be used for producing electrochemical cells, such as those present in batteries, having a more substantial lifetime.

This is accomplished by virtue of an ionic liquid having a color of less than 115 Hazen units on the APHA scale. It has been found that, surprisingly, an ionic liquid based on an FSI anion and an onium cation and having a color value of strictly less than 115 Hazen units is able to have electrochemical performance as required for the use thereof in Li-ion batteries. Without wishing to be tied to any theory, it is thought that when the ionic liquid has a color of greater than 115 Hazen units, the colored impurities present in the ionic liquid and responsible for the color of the ionic liquid provoke secondary reactions when the ionic liquid is used in an electrolyte. These secondary reactions are characterized by a high oxidation current in the Li-ion batteries, so giving rise to a decrease in their capacity over time. Removing at least a portion of the colored impurities can reduce the secondary reactions, thereby improving the lifetime of the batteries.

The present invention also provides a process capable of obtaining an ionic liquid displaying the advantages referred to above.

This is accomplished by virtue of the combination of activated carbon decolorization and at least one aqueous washing of the ionic liquid. The reason is that treating the ionic liquid using activated carbon is able to reduce the level of colored impurities but introduces impurities into the ionic liquid. These impurities are then removed or reduced by virtue of the aqueous washing of the ionic liquid.

### BRIEF DESCRIPTION OF THE FIGURES

FIG. 1 represents the flash points (on the y-axis, in ° C.) of the compositions described in example 1 as a function of the mass proportions of ionic liquid EMIM:FSI in the composition (on the x-axis, in % by mass).

FIG. 2 represents the ionic conductivity (on the y-axis, in mS/cm) of the electrolytes described in example 1 as a function of the mass proportion x of ionic liquid EMIM:FSI in the composition (on the x-axis, in % by mass), for an electrolyte comprising (100-x)% of an EC/EMC mixture at 3/7 by volume and a concentration of 0.7 mol/L of LiFSI (curve A), for an electrolyte comprising (100-x)% of an EC/EMC mixture at 3/7 by volume and a concentration of 0.8 mol/L of LiFSI (curve B), for an electrolyte comprising (100-x)% of an EC/EMC mixture at 3/7 by volume and a concentration of 0.9 mol/L of LiFSI (curve C) and for an electrolyte comprising (100-x)% of an EC/EMC mixture at 3/7 by volume and a concentration of 1 mol/L of LiFSI (curve D).

### DETAILED DESCRIPTION

The invention is now described in more detail and in a nonlimiting way in the description which follows.

Unless otherwise indicated, all the percentages and proportions are mass percentages and proportions and all the ratios between two quantities are mass ratios.

### Ionic Liquid

The invention relates first to an ionic liquid comprising an anion of formula (I):

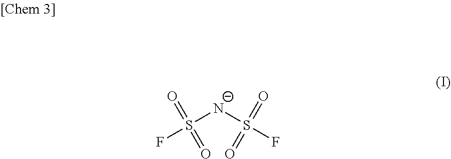

and one or more onium cations.

Ionic liquids are salts which possess a melting temperature of less than 100° C. and preferably less than room temperature (i.e. than a temperature ranging from 15 to 35° C.). Accordingly, an “ionic liquid” refers to a salt, i.e. an ionic compound comprising at least an anion and a cation, which is present in a liquid form at the temperature of 100° C. An ionic liquid comprises solely ionic species (cations and anions), except for the possible presence of nonionic impurities. Accordingly, in the sense of the present invention, an ionic liquid comprises at least 90% by weight, preferably at least 95% by weight, more preferably at least 98% by weight, more preferably at least 99% by weight, still more preferably at least 99.5% by weight, even more preferably greater than or equal to 99.9% by weight, of ionic species.

The anion of formula (I) is the bis(fluorosulfonyl)imide anion, also called FSI anion.

The ionic liquid according to the invention comprises as cation at least one onium ion. The onium ion is preferably selected from the group consisting of quaternary ammonium ions, pyridinium ions, imidazolium ions, oxazolidinium ions, piperidinium ions, phosphonium ions and mixtures thereof.

The quaternary ammonium ion is advantageously an ion of formula NR₄⁺, in which R represents an alkyl chain of 1 to 14 carbon atoms comprising optionally one or more heteroatoms such as the heteroatoms N, O, S and/or Si.

A “pyridinium ion” means the ion of formula C₅H₅NH⁺ and its derivatives, namely the ions of formula C₅H₅NH⁺ in which one or more hydrogen atoms are substituted by a group, preferably an alkyl chain comprising optionally one or more heteroatoms such as the heteroatoms N, O, S and/or Si, more preferably comprising from 1 to 14 carbon atoms.

An “imidazolium ion” means the ion of formula C₃H₅N₂⁺ and its derivatives, namely the ions of formula C₃H₅N₂⁺ in which one or more hydrogen atoms are substituted by a group, preferably an alkyl chain comprising optionally one or more heteroatoms such as the heteroatoms N, O, S and/or Si, more preferably comprising from 1 to 14 carbon atoms.

An “oxazolidinium ion” means the ion of formula C₃H₈NO⁺ and its derivatives, namely the ions of formula C₃H₈NO⁺ in which one or more hydrogen atoms are substituted by a group, preferably an alkyl chain comprising optionally one or more heteroatoms such as the heteroatoms N, O, S and/or Si, more preferably comprising from 1 to 14 carbon atoms.

A “piperidinium ion” means the ion of formula C₅H₁₂N⁺ and its derivatives, namely the ions of formula C₅H₁₂N⁺ in which one or more hydrogen atoms are substituted by a group, preferably an alkyl chain comprising optionally one or more heteroatoms such as the heteroatoms N, O, S and/or Si, more preferably comprising from 1 to 14 carbon atoms.

A “phosphonium ion” means the ion of formula PR′₄⁺, in which R′ represents an alkyl chain, preferably of 1 to 14 carbon atoms, comprising optionally one or more heteroatoms such as the heteroatoms N, O, S and/or Si.

The ionic liquid may consist essentially of the anion of formula (I) and the one (or more) onium cation(s), meaning that the anion of formula (I) and the one (or more) onium cation(s) may be present in an amount of greater than or equal to 90% by weight, preferably greater than or equal to 95% by weight, more preferably greater than or equal to 98% by weight, more preferably greater than or equal to 99% by weight, still more preferably greater than or equal to 99.5% by weight, even more preferably greater than or equal to 99.9% by weight, relative to the total weight of the ionic liquid.

In some embodiments, the ionic liquid may comprise one or more other anions and/or one or more other cations.

The ionic liquid of the present invention may, further to the anion of formula (I), comprise at least one other anion chosen from Cl⁻, Br⁻, I⁻, NO₃⁻, M(R¹)₄⁻, A(R¹)₆⁻, R²O₂⁻, [R²ONZ¹]⁻, [R²YOCZ²Z³]—, 4,5-dicyano-1,2,3-triazole, 3,5-bis(R_{F})-1,2,4-triazole, tricyanomethane, pentacyanocyclopentadiene, pentakis(trifluoromethyl)cyclopentadiene, derivatives of barbituric acid and of Meldrum's acid, and substitution products thereof;

in which
M is B, Al, Ga or Bi;
A is P, As or Sb;
R¹ is a halogen;
R² represents H, F, an alkyl, alkenyl, aryl, arylalkyl, alkylaryl, arylalkenyl, alkenylaryl, dialkylamino, alkoxy or thioalkoxy group, each having from 1 to 18 carbon atoms and being unsubstituted or substituted by one or more oxa, thia or aza substituents, and in which one or more hydrogen atoms are optionally replaced by a halogen in a proportion of 0 to 100%, and which may optionally form part of a polymer chain;
Y represents C, SO, S═NCN, S═C(CN)₂, POR², P(NCN)R², P(C(CN)₂)R2, an alkyl, alkenyl, aryl, arylalkyl, alkylaryl, arylalkenyl or alkenylaryl group possessing from 1 to 18 carbon atoms and optionally substituted by one or more oxa, thia or aza substituents; or a dialkylamino group N(R¹)²;
Z¹ to Z³ represent independently R², R²YO or CN, it being possible for this group optionally to form part of a polymer chain.

Advantageously, the ionic liquid comprises F⁻ ions, in an amount of 0 to 20 ppm, and/or Cl⁻ ions, in an amount of 0 to 20 ppm, and/or SO₄²⁻ ions, in an amount of 0 to 50 ppm, and/or Na⁺ ions, in an amount of 0 to 20 ppm, and/or K⁺ ions, in an amount of 0 to 20 ppm.

The ionic liquid according to the invention has a color of less than 115 Hazen units on the APHA scale (also called Hazen scale, platinum-cobalt scale or Pt—Co scale). The color of the ionic liquid may be determined by spectrophotometric measurement according to standard ISO 6271:2015.

More preferably, the ionic liquid has a color, on the APHA scale, of less than or equal to 100 Hazen units, more preferably less than or equal to 75 Hazen units, more preferably less than or equal to 50 Hazen units, more preferably less than or equal to 25 Hazen units and even more preferably less than or equal to 20 Hazen units. In some embodiments, the color of the ionic liquid may amount, on the APHA scale, to from 1 to 5 Hazen units, or from 5 to 10 Hazen units, or from 10 to 15 Hazen units, or from 15 to 20 Hazen units, or from 20 to 25 Hazen units, or from 25 to 30 Hazen units, or from 30 to 35 Hazen units, or from 35 to 40 Hazen units, or from 40 to 45 Hazen units, or from 45 to 50 Hazen units, or from 50 to 60 Hazen units, or from 60 to 70 Hazen units, or from 70 to 80 Hazen units, or from 80 to 90 Hazen units, or from 90 to 100 Hazen units, or from 100 to 110 Hazen units, or from 110 to less than 115 Hazen units.

### Process for Preparing the Ionic Liquid

The ionic liquid comprising an anion of formula (I) and at least one onium cation may be prepared by a process comprising the following steps:
supplying a salt of the FSI anion;
supplying a salt of the onium cation;
combining the salt of the FSI anion and the salt of the onium cation, to give an ionic liquid comprising the FSI anion and the onium cation.

Thus the ionic liquid may be synthesized by an exchange reaction according to the following scheme:

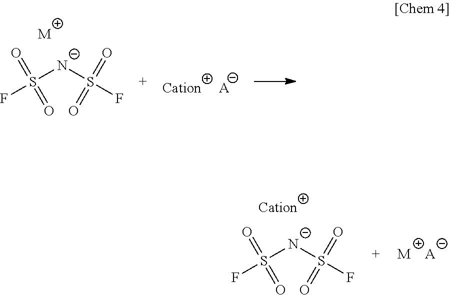

in which Cation^{⊕} is the onium cation, M^{⊕} is a cation and A^{⊖} is an anion.

M^{⊕} may in particular represent a hydrogen cation or an alkali metal or alkaline earth metal cation or a quaternary ammonium cation. It may be, for example, the hydrogen, lithium, sodium, potassium or ammonium (NH₄⁺) cation.

A^{⊖} may for example be a Cl⁻, Br⁻, BF₄⁻, F⁻, CH3COO⁻, OH⁻, NO₃⁻ or I⁻ anion or a sulfonate anion.

The reaction may be carried out for example in water, in a polar organic solvent or in a mixture of polar organic solvents.

Preferably, therefore, the salt of the FSI anion is supplied in the form of a solution of the salt of the FSI anion in water, in an organic solvent, for example nitromethane, or in a mixture of polar organic solvents.

Preferably, the salt of the onium cation is supplied in the form of a solution of the salt of the onium cation in water, in a polar organic solvent, for example nitromethane, or in a mixture of polar organic solvents.

The ionic liquid comprising the FSI anion and the onium cation may be purified, to remove the anionic A^{⊖} and cationic M^{⊕} impurities.

For example, the process may comprise a step of dissolving the combination of the salt of the FSI anion and the salt of the onium cation in an organic solvent, such as butyl acetate, and one or more steps of washing using an aqueous solution, preferably water, the ionic liquid being present in the organic phase and the anionic A^{⊖} and cationic M^{⊕} impurities being present in the aqueous phase. The organic phase may then be evaporated, preferably under reduced pressure, to recover the ionic liquid.

Before the combination of the salt of the FSI anion and the salt of the onium cation is dissolved in an organic solvent, said combination may be subjected to an evaporation step, preferably under reduced pressure, to remove the reaction solvent.

Before or after the combination of the salt of the FSI anion and the salt of the onium cation is dissolved in an organic solvent, said combination may undergo filtration, on a PTFE (polytetrafluoroethylene) membrane, for example.

### Process for Purifying the Ionic Liquid

The invention also relates to a process for purifying an ionic liquid. This process comprises the following steps:
supplying an initial ionic liquid comprising an anion of formula (I):

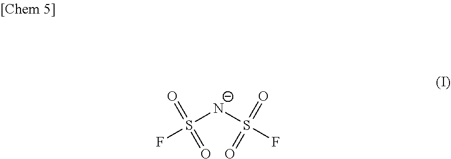

and an onium cation;
contacting said initial ionic liquid with activated carbon, to collect a decolorized ionic liquid;
at least once aqueously washing the decolorized ionic liquid;
collecting a purified ionic liquid.

By such a process, the amount of colored impurities in the ionic liquid can be reduced.

A “decolorized ionic liquid” means an ionic liquid having a color as measured on the APHA scale which is lower than that of the ionic liquid before the step of contacting the ionic liquid with the activated carbon.

A “purified ionic liquid” means an ionic liquid in which the ratio of the molar concentrations [liquid salt of the anion of formula (I) and of the onium cation]/[total impurities] is greater than that of the decolorized ionic liquid before washing.

The onium cation may be as described in the preceding sections.

The initial ionic liquid may consist essentially of the anion of formula (I) and the onium cation and/or may comprise one or more other anions and/or one or more other cations, as is described in the preceding sections.

The purified ionic liquid preferably has a color of less than 115 Hazen units on the APHA scale. The color of the ionic liquid may be determined as described above. More preferably, the purified ionic liquid has a color, on the APHA scale, of less than or equal to 100 Hazen units, more preferably less than or equal to 75 Hazen units, more preferably less than or equal to 50 Hazen units, more preferably less than or equal to 25 Hazen units and even more preferably less than or equal to 20 Hazen units. In particular, the color of the purified ionic liquid may amount, on the APHA scale, to from 1 to 5 Hazen units, or from 5 to 10 Hazen units, or from 10 to 15 Hazen units, or from 15 to 20 Hazen units, or from 20 to 25 Hazen units, or from 25 to 30 Hazen units, or from 30 to 35 Hazen units, or from 35 to 40 Hazen units, or from 40 to 45 Hazen units, or from 45 to 50 Hazen units, or from 50 to 60 Hazen units, or from 60 to 70 Hazen units, or from 70 to 80 Hazen units, or from 80 to 90 Hazen units, or from 90 to 100 Hazen units, or from 100 to 110 Hazen units, or from 110 to less than 115 Hazen units.

The color of the decolorized ionic liquid is preferably as described above.

The initial ionic liquid may be obtained as described in the preceding section.

The initial ionic liquid preferably has a color of greater than or equal to 115 Hazen units on the APHA scale. It may have a color of greater than or equal to 120 Hazen units, or 140 Hazen units, or 160 Hazen units, or 180 Hazen units, or 200 Hazen units, or 220 Hazen units, or 240 Hazen units, or 260 Hazen units, or 280 Hazen units, or 300 Hazen units, or 320 Hazen units, or 340 Hazen units, or 360 Hazen units, or 380 Hazen units, or 400 Hazen units, on the APHA scale.

The initial ionic liquid may be supplied in solution in a polar organic solvent (or a mixture of polar organic solvents). The polar organic solvent may be from the class of esters, ethers, nitriles, carbonates, ketones or combinations thereof. Examples of polar organic solvents suitable for the process according to the invention are butyl acetate, ethyl acetate, tert-butyl acetate, acetonitrile, butyronitrile, isobutyronitrile, glutaronitrile, diethyl ether, cyclopentyl methyl ether, tetrahydrofuran, methyl isobutyl ketone, dimethyl carbonate, diethyl carbonate, ethyl methyl carbonate, ethylene carbonate and propylene carbonate. The mass ratio of the ionic liquid to the polar organic solvent may advantageously amount to from 0.001 to 100, preferably from 0.01 to 10, for example from 0.001 to 0.01, or from 0.01 to 0.1, or from 0.1 to 1, or from 1 to 10, or from 10 to 100.

The initial ionic liquid may alternatively be contacted with the activated carbon alone, i.e., without prior combination with any solvent.

The contacting of the initial ionic liquid with the activated carbon may for example be carried out by mixing the activated carbon in the ionic liquid. The mass ratio of the activated carbon to the ionic liquid is advantageously from 0.05 to 0.5, preferably from 0.1 to 0.5. In particular, this ratio may amount to from 0.05 to 0.1, or from 0.1 to 0.2, or from 0.2 to 0.3, or from 0.3 to 0.4, or from 0.4 to 0.5.

The activated carbon preferably possesses a specific surface area of more than 300 m²/g, such as more than 350 m²/g, or more than 400 m²/g, or more than 500 m²/g, or more than 600 m²/g, or more than 800 m²/g, or more than 1000 m²/g. The specific surface area of the activated carbon may be measured by the BET method. The specific surface area of a powder is estimated from the amount of nitrogen adsorbed relative to its pressure at the boiling point of liquid nitrogen and under standard atmospheric pressure. The data are interpreted according to the model of Brunauer, Emmett and Teller (BET method).

The contact time of the ionic liquid with the activated carbon may amount to from 1 to 72 h, preferably from 5 to 48 h. In some embodiments, the contact time of the ionic liquid with the activated carbon amounts to from 1 to 5 h, or from 5 to 12 h, or from 12 to 24 h, or from 24 to 36 h, or from 36 to 48 h, or from 48 to 72 h.

The step of contacting the initial ionic liquid with the activated carbon may be carried out at a temperature ranging from 10° C. to less than the boiling temperature of the polar organic solvent, when the initial ionic liquid is in solution in a polar organic solvent, for example at room temperature (i.e., from 15 to 35° C.).

The step of contacting the initial ionic liquid with the activated carbon is advantageously carried out at a temperature greater than the melting temperature of the ionic liquid when the initial ionic liquid is in contact with the activated carbon alone.

At the end of the activated carbon treatment step, the activated carbon is advantageously separated from the decolorized ionic liquid by filtration, for example using a PTFE membrane, or through a poly(vinylidene fluoride) (PVDF) membrane, or through a cellulosic membrane, or through a filtering medium (silica, alumina, diatomaceous earth).

When the ionic liquid which has been contacted with the activated carbon is in solution in a polar organic solvent, this solvent can be removed after the activated carbon contacting step, or preferably after the separation of the activated carbon from the decolorized ionic liquid. The removal may be done for example by evaporation of the solvent, preferably under reduced pressure. Alternatively, the polar organic solvent is not removed (for example if this solvent is insoluble in water).

A “water-insoluble solvent” means a solvent whose solubility in water at 25° C. is less than 10% by weight. The solubility of the solvent may be determined by gradually adding said solvent to a mass of water until separation is observed.

The decolorized ionic liquid is subjected to one or more aqueous washes. “Aqueous wash” or “aqueous washing” means the contacting of the ionic liquid with an aqueous solution, preferably water, more preferentially demineralized water.

With particular preference, the ionic liquid undergoing the aqueous wash or washes is in solution in a water-insoluble polar organic solvent. In particular, when the ionic liquid has been contacted with the activated carbon without being in solution in a solvent, or when the solvent has been removed after the activated carbon contacting step, the ionic liquid is dissolved in a water-insoluble polar organic solvent. Alternatively, the water-insoluble polar organic solvent in which the ionic liquid is dissolved to undergo aqueous washing may be the polar organic solvent in which the ionic liquid was dissolved for the activated carbon contacting step. The water-insoluble polar organic solvent is selected from the group consisting of butyl acetate, ethyl acetate, tert-butyl acetate, butyronitrile, isobutyronitrile, glutaronitrile, diethyl ether, cyclopentyl methyl ether, tetrahydrofuran, methyl isobutyl ketone, dimethyl carbonate, diethyl carbonate, ethyl methyl carbonate, ethylene carbonate and propylene carbonate.

The aqueous wash or washes make it possible to reduce and remove the impurities present in the decolorized ionic liquid, and in particular the impurities generated by the step of treating the ionic liquid with the activated carbon, such as chloride ions, fluoride ions, sodium ions and/or potassium ions.

During each aqueous wash, the mass ratio of the aqueous washing solution, preferably the demineralized water, to the ionic liquid amounts to preferably from 0.01 to 1, for example from 0.01 to 0.05, or from 0.05 to 0.1, or from 0.1 to 0.5, or from 0.5 to 1.

The contact time between the ionic liquid and the aqueous washing solution may vary from 10 min to 5 h. In particular, this time may be from 10 to 30 min, or from 30 min to 1 h, or from 1 h to 2 h, or from 2 h to 3 h, or from 3 h to 4 h, or from 4 h to 5 h.

The aqueous phase is then advantageously separated from the organic phase by decanting. The organic phase is enriched in the ionic liquid and depleted in impurities (for example depleted in chloride, fluoride, sodium and/or potassium ions), meaning that in the organic phase, the ratio of the molar concentrations of ionic liquid/impurities (more particularly chloride, fluoride, sodium and/or potassium ions) is greater than that of the decolorized ionic liquid. The aqueous phase is enriched in impurities (for example enriched in chloride, fluoride, sodium and/or potassium ions), meaning that in the aqueous phase, the ratio of the molar concentrations of ionic liquid/impurities (more particularly chloride, fluoride, sodium and/or potassium ions) is lower than that of the decolorized ionic liquid.

The aqueous phase may then be removed.

A number of aqueous washes may be performed, in particular from 2 to 11 aqueous washes (for example, two, or three, or four, or five, or ten washes). When a number of washes are performed, each independently may be as described above. The subsequent wash is preferably carried out on the organic phase obtained, after decanting, at the end of the previous wash.

The solvent of the organic phase may be removed, for example by evaporation of the solvent, preferably under reduced pressure. A purified ionic liquid is obtained.

The invention also relates to an ionic liquid obtained or obtainable by the process as described above.

### Electrochemical Cell and Battery

The invention also relates to an electrolyte comprising an ionic liquid as described above and at least one other component chosen from metal salts, polar polymers and/or aprotic solvents.

The metal salt preferably comprises as cation the hydrogen cation, the cation of an alkali metal, of an alkaline earth metal, of a transition metal or of a rare earth, with lithium being especially preferred.

By way of nonlimiting examples, the lithium salt (or the lithium salts) can be chosen from LiPF₆ (lithium hexafluorophosphate), LiFSI (lithium bis(fluorosulfonyl)imide), LiTDI (lithium 2-trifluoromethyl-4,5-dicyanoimidazolate), LiPOF₂, LiB(C₂O₄)₂, LiF₂B(C₂O₄)₂, LiBF₄, LiNO₃ or LiClO₄.

The polar polymer preferably comprises monomer units derived from ethylene oxide, propylene oxide, epichlorohydrin, epifluorohydrin, trifluoroepoxypropane, acrylonitrile, methacrylonitrile, esters and amides of acrylic and methacrylic acid, vinylidene fluoride, N-methylpyrrolidone and/or polycation or polyanion polyelectrolytes. When the present electrolyte composition comprises more than one polymer, at least one of them may be crosslinked.

The aprotic solvent or solvents can be chosen from the following nonexhaustive list: ethers, esters, ketones, alcohols, nitriles, carbonates, amides, sulfamides and sulfonamides and their mixtures.

Mention may be made, among the ethers, of linear or cyclic ethers, such as, for example, dimethoxyethane (DME), methyl ethers of oligoethylene glycols of 2 to 5 oxyethylene units, dioxolane, dioxane, dibutyl ether, tetrahydrofuran and their mixtures.

Mention may be made, among the esters, of phosphoric acid esters or sulfite esters. Mention may be made, for example, of methyl formate, methyl acetate, methyl propionate, ethyl acetate, butyl acetate, gamma-butyrolactone or their mixtures.

Mention may in particular be made, among the ketones, of cyclohexanone.

Mention may be made, among the alcohols, for example, of ethyl alcohol or isopropyl alcohol.

Mention may be made, among the nitriles, for example, of acetonitrile, pyruvonitrile, propionitrile, methoxypropionitrile, dimethylaminopropionitrile, butyronitrile, isobutyronitrile, valeronitrile, pivalonitrile, isovaleronitrile, glutaronitrile, methoxyglutaronitrile, 2-methylglutaronitrile, 3-methylglutaronitrile, adiponitrile, malononitrile, 1,2,6-tricyanohexane and mixtures thereof.

Mention may be made, among the carbonates, for example, of cyclic carbonates such as, for example, ethylene carbonate (EC) (CAS: 96-49-1), propylene carbonate (PC) (CAS: 108-32-7), butylene carbonate (BC) (CAS: 4437-85-8), dimethyl carbonate (DMC) (CAS: 616-38-6), diethyl carbonate (DEC) (CAS: 105-58-8), ethyl methyl carbonate (EMC) (CAS: 623-53-0), diphenyl carbonate (CAS: 102-09-0), methyl phenyl carbonate (CAS: 13509-27-8), dipropyl carbonate (DPC) (CAS: 623-96-1), methyl propyl carbonate (MPC) (CAS: 1333-41-1), ethyl propyl carbonate (EPC), vinylene carbonate (VC) (CAS: 872-36-6), fluoroethylene carbonate (FEC) (CAS: 114435-02-8), trifluoropropylene carbonate (CAS: 167951-80-6) or their mixtures.

Mention may be made, among the amides, of dimethylformamide and N-methylpyrrolidinone.

The aprotic solvent is chosen more preferably from EC, EMC, mixtures of EC and EMC, mixtures of EC and DMC, mixtures of EC and DEC, PC, mixtures of EC, DMC and EMC.

The electrolyte preferably comprises or consists of the ionic liquid as described above, one or more lithium salts (for example as recited above) dissolved in a solvent or a mixture of solvents (for example as recited above), with optionally one or more additives.

The additive or additives may be selected from the group consisting of fluoroethylene carbonate (FEC), vinylene carbonate, 4-vinyl-1,3-dioxolan-2-one, pyridazine, vinylpyridazine, quinoline, vinylquinoline, butadiene, sebaconitrile, alkyl disulfides, fluorotoluene, 1,4-dimethoxytetrafluorotoluene, t-butylphenol, di(t-butyl)phenol, tris(pentafluorophenyl)borane, oximes, aliphatic epoxides, halogenated biphenyls, methacrylic acids, allyl ethyl carbonate, vinyl acetate, divinyl adipate, propane sultone, acrylonitrile, 2-vinylpyridine, maleic anhydride, methyl cinnamate, phosphonates, silane compounds containing a vinyl, and/or 2-cyanofuran.

Advantageously, the ionic liquid is present in the electrolyte in an amount of 10% to 90% by weight, preferably from 20% to 80% by weight, more preferentially from 40% to 80% by weight, relative to the total weight of the electrolyte. In some embodiments, the electrolyte may comprise from 10% to 20%, or from 20% to 30%, or from 30% to 40%, or from 40% to 50%, or from 50% to 60%, or from 60% to 70%, or from 70% to 80%, or from 80% to 90%, by weight, of ionic liquid (relative to the total weight of the electrolyte).

The invention also relates to an electrochemical cell comprising an electrolyte comprising an ionic liquid as described above. The electrochemical cell also comprises a negative electrode (or anode) and a positive electrode (or cathode).

The electrochemical cell can also comprise a separator, in which the electrolyte is impregnated.

The electrolyte can be as described above.

A “negative electrode” means the electrode which acts as anode when the cell delivers current (that is to say, when it is in the process of discharging) and which acts as cathode when the cell is in the process of charging.

The negative electrode typically comprises an electrochemically active material, optionally an electronically conductive material, and optionally a binder.

A “positive electrode” means the electrode which acts as cathode when the cell delivers current (that is to say, when it is in the process of discharging) and which acts as anode when the cell is in the process of charging.

The positive electrode typically comprises an electrochemically active material, optionally an electronically conductive material, and optionally a binder.

The term “electrochemically active material” is understood to mean a material capable of reversibly inserting ions.

The term “electronically conductive material” is understood to mean a material capable of conducting electrons.

The negative electrode of the electrochemical cell can in particular comprise, as electrochemically active material, graphite, lithium, a lithium alloy, a lithium titanate of Li₄Ti₅O₁₂ type or titanium oxide TiO₂, silicon or a lithium-silicon alloy, a tin oxide, a lithium intermetallic compound, or a mixture thereof.

When the negative electrode comprises lithium, the latter can be in the form of a film of metallic lithium or of an alloy comprising lithium. Mention may be made, for example, among the lithium-based alloys capable of being used, of lithium-aluminum alloys, lithium-silica alloys, lithium-tin alloys, Li—Zn, Li₃Bi, Li₃Cd and Li₃SB. An example of negative electrode can comprise an active lithium film prepared by rolling a strip of lithium between rollers.

The positive electrode comprises an electrochemically active material of oxide type. It is preferably a lithium-iron phosphate (LiₓFePO₄ with 0<x<1), or a lithium/nickel/manganese/cobalt composite oxide having a high nickel content (LiNiₓMn_{y}Co_{z}O₂ with x+y+z=1, abbreviated to NMC, with x>y and x>z), or a lithium/nickel/cobalt/aluminum composite oxide having a high nickel content (LiNi_{x′}Co_{y′}Al_{z′} with x′+y′+z′=1, abbreviated to NCA, with x′>y′ and x′>z′).

Specific examples of these oxides are NMC532 (LiNi_{0.5}Mn_{0.3}Co_{0.2}O₂), NMC622 (LiNi_{0.6}Mn_{0.2}Co_{0.2}O₂) and NMC811 (LiNi_{0.8}Mn_{0.1}Co_{0.1}O₂).

Mixtures of these oxides can be used. The oxide material described above can, if appropriate, be combined with another oxide, such as, for example: manganese dioxide (MnO₂), iron oxide, copper oxide, nickel oxide, lithium/manganese composite oxides (for example LiₓMn₂O₄ or LiₓMₙO₂), lithium/nickel oxide compositions (for example LiₓNiO₂), lithium/cobalt oxide compositions (for example LiₓCoO₂), lithium/nickel/cobalt composite oxides (for example LiNi_{1-y}Co_{y}O₂), lithium and transition metal composite oxides, lithium/manganese/nickel composite oxides of spinel structure (for example LiₓMn_{2-y}Ni_{y}O₄), vanadium oxides, NMC and NCA oxides which do not have a high nickel content and their mixtures.

Preferably, the NMC or NCA oxide having a high nickel content represents at least 50% by weight, preferably at least 75% by weight, more preferably at least 90% by weight and more preferably essentially all of the oxide material present in the positive electrode as electrochemically active material.

Alternatively or additionally, the positive electrode may comprise sulfur, Li₂S, O₂, and/or LiO₂ as electrochemically active material.

The material of each electrode can also comprise, besides the electrochemically active material, an electronically conductive material, such as a carbon source, including, for example, carbon black, Ketjen® carbon, Shawinigan carbon, graphite, graphene, carbon nanotubes, carbon fibers (for example, vapor-grown carbon fibers or VGCF), non-powdery carbon obtained by carbonization of an organic precursor, or a combination of two or more of these. Other additives can also be present in the material of the positive electrode, such as lithium salts or inorganic particles of ceramic or glass type, or also other compatible active materials (for example, sulfur).

The material of each electrode can also comprise a binder. Nonlimiting examples of binders comprise linear, branched and/or crosslinked polyether polymer binders (for example polymers based on poly(ethylene oxide) (PEO), or poly(propylene oxide) (PPO) or on a mixture of the two (or an EO/PO copolymer), and optionally comprising crosslinkable units), water-soluble binders (such as SBR (styrene/butadiene rubber), NBR (acrylonitrile/butadiene rubber), HNBR (hydrogenated NBR), CHR (epichlorohydrin rubber), ACM (acrylate rubber)), or binders of fluoropolymer type (such as PVDF (polyvinylidene fluoride), PTFE (polytetrafluoroethylene)), and combinations thereof. Some binders, such as those which are soluble in water, can also comprise an additive, such as CMC (carboxymethylcellulose).

The separator can be a porous polymer film. By way of nonlimiting example, the separator can consist of a porous film of polyolefin, such as ethylene homopolymers, propylene homopolymers, ethylene/butene copolymers, ethylene/hexene copolymers, ethylene/methacrylate copolymers or multilayer structures of the above polymers. Alternatively, the separator may be made of glass fibers.

The invention also relates to a battery comprising at least one, and preferably two or more, electrochemical cells as described above. The electrochemical cells can be assembled in series and/or in parallel in the battery.

### Other Applications

The ionic liquid according to the invention may also be used in an electrolyte in an electrochromic light modulation system comprising at least one electrochromic material. In such a system, the electrochromic material is advantageously deposited on a layer of a semiconductor transparent in the visible range, preferably a tin oxide or indium oxide derivative, on a glass or polymer substrate. Examples of preferential electrochromic materials include molybdenum, tungsten, titanium, vanadium, niobium, cerium and tin oxides, and also mixtures thereof. The electrochromic material may optionally be dissolved in the electrolyte.

The ionic liquid according to the invention may also be used in a composition as a reaction medium for chemical or electrochemical reactions, preferably for Diels-Alder, Friedel-Craft, mixed aldolization, condensation and polymerization reactions, and for nucleophilic and electrophilic substitutions. When the ionic liquid comprises a chiral onium cation, the ionic liquid may be used in a composition as a reaction medium for enantioselective reactions.

The ionic liquid according to the invention may also be used for treatment of a surface, for example for cleaning this surface.

### EXAMPLES

The following examples illustrate the invention without limiting it.

### Example 1—Effect of the Presence of Ionic Liquid in an Electrolyte

### Flash Point

An ionic liquid of 1-ethyl-3-methylimidazolium bis(fluorosulfonyl)imide (EMIM:FSI), having a color of 20 Hazen units, was prepared as described in example 2.

The EMIM:FSI was added in different mass proportions to a mixture of carbonates (EC/EMC 3/7 v/v) conventionally used in Li-ion battery electrolytes.

For each proportion of EMIM:FSI, the flash point of the composition was measured according to standard ISO 3679.

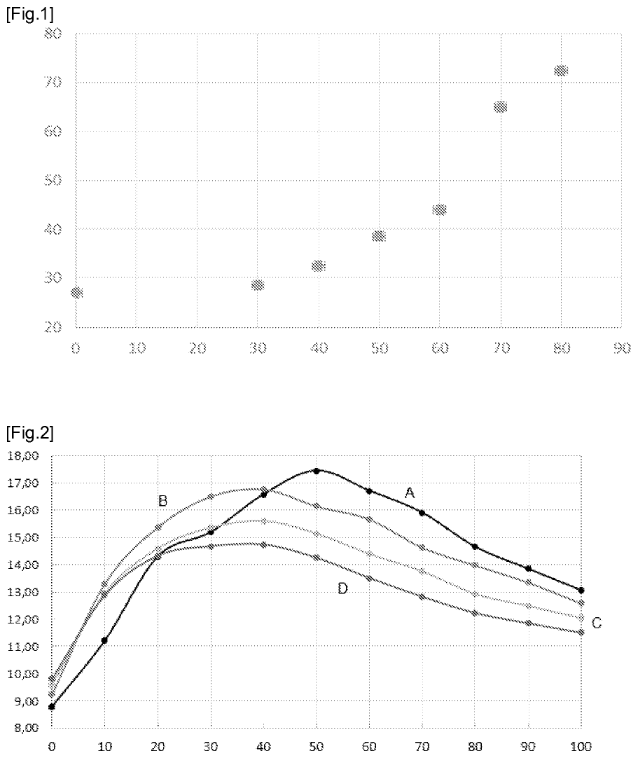

The results are shown in FIG. 1.

It is observed that the addition of ionic liquid EMIM:FSI to the composition raises the flash point and may thus suppress the flammability of battery electrolytes. Moreover, for levels of EMIM:FSI of greater than or equal to 70%, the compositions attain a sufficient flash point allowing them to change category in the context of transport regulations (the upper flash point limit for flammable products, according to transport regulations, being set at 60° C.). For such amounts, it is therefore important that the ionic liquid has good electrochemical stability.

### Ionic Conductivity

Different electrolytes were prepared, these electrolytes comprising different mass proportions of ionic liquid EMIM:FSI in a mixture of carbonates (EC/EMC 3/7 v/v) also comprising different concentrations of LiFSI (0.7 mol/L; 0.8 mol/L; 0.9 mol/L; 1 mol/L).

For each of the electrolytes thus prepared, the ionic conductivity of the electrolyte was determined by impedance spectroscopy measurements. For this, a conductivity cell was immersed in each of the solutions and three impedance spectroscopy determinations were carried out. These spectroscopy determinations are carried out between 500 mHz and 100 kHz with an amplitude of 10 mV. The constant of the cell used is 1.12 and the ionic conductivity a is calculated according to the following formula:

$$σ = \frac{1}{R} \times 1.12 \qquad [ Math 1 ]$$

where R represents the resistance which is obtained by linear regression of the curve Im(Z)=f(Re(Z)). In the specific case of Im(Z)=0, R is equal to the opposite of the ordinate at the origin divided by the slope of the linear regression equation.

The results are shown in FIG. 2.

It is observed that the addition of EMIM:FSI to the electrolytes enables a significant increase in the ionic conductivity of the electrolyte. Moreover, optimum conductivities are obtained for substantial proportions of ionic liquid. It is therefore vital to use an ionic liquid of good electrochemical stability and thus high purity.

This example demonstrates the importance of having an ionic liquid available that has high purity, in view of the very large amounts of ionic liquid the electrolyte must contain to have better conductivity and lower flammability.

### Example 2—Preparation of an Ionic Liquid EMIM:FSI

In a 250 mL reactor, 30 g of 1-ethyl-3-methylimidazolium chloride are dissolved in 60 g of nitromethane. When the reaction mixture becomes homogeneous, a solution of potassium bis(fluorosulfonyl)imide (42.73 g) in 120 g of nitromethane is added. The reaction mixture is left with stirring for 12 hours.

The reaction mixture is filtered on a 0.45 μm PTFE membrane. The filtrate is then evaporated under reduced pressure to remove the residual solvent.

The residue obtained is then diluted in 50 g of butyl acetate. This solution is then contacted with 25 g of water. After decanting, the organic phase containing the ionic liquid is recovered and the aqueous phase is discarded. This washing is carried out three times with the same amount of water. The organic phase is then evaporated under reduced pressure to recover the ionic liquid with a yield of 71% (39.29 g). The color of the ionic liquid obtained is 115 Hazen units. The color of the ionic liquid obtained is measured using a Lico spectral colorimeter according to standard ISO 6271:2015.

The ionic liquid obtained before is dissolved in 80 g of butyl acetate. Activated carbon (6 g) is added and the solution is left with stirring for 4 hours. The carbon is then removed by filtration on a 0.45 pm PTFE membrane and rinsed with three times 20 g of butyl acetate. The filtrate is then evaporated under reduced pressure to recover the ionic liquid with a yield of 96.65%. The color of the ionic liquid after purification is 20 Hazen units. However, the ionic liquid contains cationic and anionic impurities such as chlorides, fluorides, sodiums and potassiums.

A further purification is then carried out. The ionic liquid is dissolved in 40 g of butyl acetate. This solution is washed with four times 20 g of water. The aqueous phases are removed and the organic phase is evaporated under reduced pressure to give 29.7 g of ionic liquid with a color of 20 Hazen units.

### Example 3—Preparation of an Ionic Liquid PYR14:FSI (1-butyl-1-methylpyrrolidinium bis(fluorosulfonyl)imide)

In a 1 L reactor, 120 g of 1-butyl-1-methylpyrrolidinium chloride are dissolved in 250 g of nitromethane. When the reaction mixture becomes homogeneous, a solution of potassium bis(fluorosulfonyl)imide (140.7 g) in 120 g of nitromethane is added. The reaction mixture is left with stirring for 24 hours.

The reaction mixture is filtered on a 0.45 μm PTFE membrane. The filtrate is then evaporated under reduced pressure to remove the residual solvent.

The residue obtained is then diluted in 200 g of butyl acetate. This solution is then contacted with 100 g of water. After decanting, the organic phase containing the ionic liquid is recovered and the aqueous phase is discarded. This washing is carried out three times with the same amount of water. The organic phase is then evaporated under reduced pressure to recover the ionic liquid with a yield of 82% (169.9 g). The color of the ionic liquid obtained is 135 Hazen units.

The ionic liquid obtained before is dissolved in 250 g of butyl acetate. Activated carbon (30 g) is added and the solution is left with stirring for 20 hours. The carbon is then removed by filtration on a 0.45 μm PTFE membrane and rinsed with three times 100 g of butyl acetate. The filtrate is then evaporated under reduced pressure to recover the ionic liquid with a yield of 94.3%. The color of the ionic liquid after purification is 20 Hazen units. However, the ionic liquid contains cationic and anionic impurities such as chlorides, fluorides, sodiums and potassiums.

A further purification is then carried out. The ionic liquid is dissolved in 250 g of butyl acetate. This solution is washed with four times 50 g of water. The aqueous phases are removed and the organic phase is evaporated under reduced pressure to give 152.2 g of ionic liquid with a color of 20 Hazen units.

### Example 4—Color Effect

Ionic liquids EMIM:FSI having a color of 115 Hazen units and 20 Hazen units were prepared as described in example 2. Further, a third ionic liquid EMIM:FSI was prepared in the same way as the ionic liquid of 115 Hazen units color but without purifying the starting materials before the synthesis (the raw materials having been purified by contacting with activated carbon and by aqueous washing to give the ionic liquids of 115 and 20 Hazen units color). This third ionic liquid has a color of 360 Hazen units.

The electrochemical stability of each of the ionic liquids is determined by cyclic voltammetry measurements. For this, CR2032 button cells are manufactured. These button cells are equipped with a 20 mm diameter aluminum foil as working electrode, an 8 mm diameter pellet of lithium metal as reference electrode and an 18 mm diameter glass fiber separator impregnated with 12 drops (0.6 mL) of an electrolyte consisting of the ionic liquid EMIM:FSI. A voltage sweep is then carried out at the terminals of the button cell and the current generated is measured and recorded. The voltage sweep is carried out between 2 and 5 V. The oxidation current is measured during the third cycle. The two prior sweeps allow the formation of passivation layers such as the SEI (solid-electrolyte interface) and the passivation of the aluminum.

Moreover, the approximate lifetime of 4 mAh batteries each comprising an electrolyte containing one of the three ionic liquids above is also measured. The lifetime is determined as being the number of cycles carried out before 80% of the initial capacity is attained. At each cycle, each battery loses a capacity equivalent to the oxidation current of the ionic liquid at 4.3 V. When this loss reaches 0.8 mAh, the battery is deemed to have reached its end of life.

The results are presented in the table below (the oxidation current in the table below is that measured at 4.3 V, this being the conventional operating voltage of Li-ion batteries).

TABLE 1

| | | | |
|---|---|---|---|
| Color of the ionic liquid (Hazen units) | 20 | 115 | 360 |
| Oxidation current (μA) at 4.3 V | 1.51 | 54.4 | 106 |
| Numbers of cycles to reach 80% of the | 530 | 15 | 8 |
| initial capacity | | | |

The color of the ionic liquid is found to have an effect on the lifetime of the battery. Indeed, an ionic liquid with a color of 20 Hazen units allows the lifetime of a battery to be multiplied 35-fold as compared to an ionic liquid with a color of 115 Hazen units.

Moreover, the oxidation current measurements show that an ionic liquid having a color of 20 Hazen units exhibits better electrochemical stability than an ionic liquid having a color of 115 Hazen units.

1. An ionic liquid comprising an anion of formula (I): 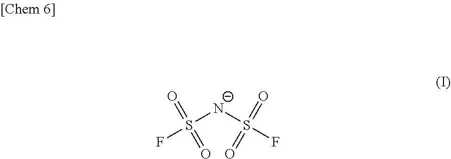
and at least one onium cation,
said ionic liquid having a color of less than 115 Hazen units on the APHA scale.

2. The ionic liquid as claimed in claim 1, wherein the onium cation is a quaternary ammonium ion, a pyridinium ion, an imidazolium ion, an oxazolidinium ion, a piperidinium ion, and/or a phosphonium ion.

3. The ionic liquid as claimed in claim 1, having a color of less than or equal to 100 Hazen units, preferably less than or equal to 75 Hazen units, more preferably less than or equal to 50 Hazen units on the APHA scale.

4. The ionic liquid as claimed in claim 1, wherein the anion of formula (I) and the onium cation are present in an amount of greater than or equal to 90% by weight relative to the total weight of the ionic liquid.

5. The ionic liquid as claimed in claim 1, further comprising from 0 to 20 ppm of F⁻ ions, from 0 to 20 ppm of Cl⁻ ions, from 0 to 50 ppm of SO₄²⁻ ions, from 0 to 20 ppm of Na⁺ ions and from 0 to 20 ppm of K⁺ ions.

6. A process for purifying an ionic liquid, comprising the following steps:
supplying an initial ionic liquid comprising an anion of formula (I): 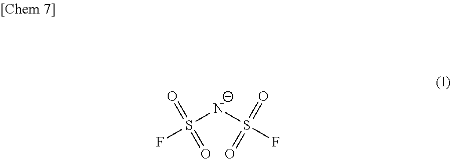
and an onium cation;
contacting said initial ionic liquid with activated carbon, to collect a decolorized ionic liquid;
at least once aqueously washing the decolorized ionic liquid;
collecting a purified ionic liquid having a color of less than 115 Hazen units on the APHA scale.

7. The process as claimed in claim 6, wherein the initial ionic liquid has a color of greater than or equal to 115 Hazen units on the APHA scale.

8. The process as claimed in claim 6, wherein the initial ionic liquid is in solution in a polar organic solvent, preferably selected from the group consisting of esters, ethers, nitriles, carbonates and mixtures thereof.

9. The process as claimed in claim 6, wherein the activated carbon has a specific surface area of greater than or equal to 300 m²/g.

10. The process as claimed in claim 6, wherein the mass ratio of the activated carbon relative to the initial ionic liquid is from 0.05 to 0.5.

11. The process as claimed in claim 6, wherein the aqueous washing comprises contacting the decolorized ionic liquid, dissolved in a water-insoluble polar organic solvent, with a mass of demineralized water.

12. An ionic liquid obtainable by the process as claimed in claim 6.

13. An electrochemical cell comprising a negative electrode, a positive electrode and an electrolyte, wherein the electrolyte comprises an ionic liquid as claimed in claim 1.

14. A battery comprising at least one electrochemical cell as claimed in claim 13.
